(19) United States
(12) Patent Application Publication
(10) Pub. No.: US 2017/0042466 A1
(43) Pub. Date: Feb. 16, 2017

(54) ELECTRONIC DEVICE, HEALTH SUPPORT SYSTEM, AND HEALTH SUPPORT METHOD

(71) Applicant: KYOCERA Corporation, Kyoto (JP)

(72) Inventors: Hirohide YAMASAKI, Yokohama-shi, Kanagawa (JP); Katsuhiro MITSUI, Kawasaki-shi, Kanagawa (JP)

(73) Assignee: KYOCERA Corporation, Kyoto (JP)

(21) Appl. No.: 15/304,396

(22) PCT Filed: Apr. 22, 2015
(86) PCT No.: PCT/JP2015/002193

§ 371 Date: Oct. 14, 2016

(30) Foreign Application Priority Data
Apr. 23, 2014 (JP) 2014-089511

(51) Int. Cl. A61B 5/00; G09B 19/00; G06F 19/00; A61B 5/026

(52) U.S. Cl. CPC A61B 5/486; A61B 5/0261; A61B 5/742; A61B 5/6898; A61B 5/7475; G09B 19/0092; G09B 19/00; G06F 19/322; G06F 19/3481

(57) ABSTRACT

Included are a biological sensor, a recorder that records a log in which an activity record of a user is associated with biological information measured by the biological sensor before and after a time pertaining to the activity record, and a controller that provides activity advice by referring to the log based on biological information measured by the biological sensor.

### CROSS-REFERENCE TO RELATED APPLICATION

This application claims priority to and the benefit of Japanese Patent Application No. 2014-089511 filed Apr. 23, 2014, the entire contents of which are incorporated herein by reference.

### TECHNICAL FIELD

This disclosure relates to an electronic device, health support system, and health support method.

### BACKGROUND

Apparatuses have been developed to support users health with a variety of methods.

### SUMMARY

An electronic device according to this disclosure includes:

a biological sensor;

a recorder configured to record a log in which an activity record of a user is associated with biological information measured by the biological sensor before and after a time pertaining to the activity record; and

a controller configured to provide activity advice by referring to the log based on biological information measured by the biological sensor.

A health support system according to this disclosure includes:

a measurement terminal comprising a biological sensor; and

a server comprising a recorder and a controller, the recorder being configured to record a log in which an activity record related to an activity is associated with biological information measured by the biological sensor before and after a time pertaining to the activity record, and the controller being configured to provide activity advice by referring to the log based on biological information measured by the biological sensor.

A health support method according to this disclosure includes:

measuring biological information with a biological sensor;

recording, with a recorder, a log in which an activity record related to a user is associated with the biological information for before and after a time pertaining to the activity record; and

providing, with a controller, activity advice by referring to the log based on measured biological information.

### BRIEF DESCRIPTION OF THE DRAWINGS

In the accompanying drawings:

FIG. 1 is a block diagram schematically illustrating the structure of an electronic device according to one of the embodiments of this disclosure;

FIGS. 2A and 2B are external views of an example of an electronic device in which a blood flow sensor is mounted;

FIG. 3 is a flowchart illustrating an example of processing to record a log in the electronic device of FIG. 1;

FIG. 4 is a flowchart illustrating an example of processing to record a log in the electronic device of FIG. 1; and

FIG. 5 is a block diagram schematically illustrating an example of a measurement terminal and a server when implementing this disclosure as a system.

### DETAILED DESCRIPTION

Known apparatuses provide an evaluation or advice based on the user's daily habits, biological information, or the like. The biological information, however, is affected by the nature and extent of the user's activities, and the biological information that is measured may change depending on the user's activity before measurement. Therefore, by recording biological information in association with the user's activities, a health support apparatus could provide more appropriate advice for improving or maintaining the user's biological information. Furthermore, advice provided in this way is highly useful for the user.

Therefore, it would be helpful to provide an electronic device, a health support system, and a health support method that provide useful health support information.

The following describes embodiments with reference to the drawings.

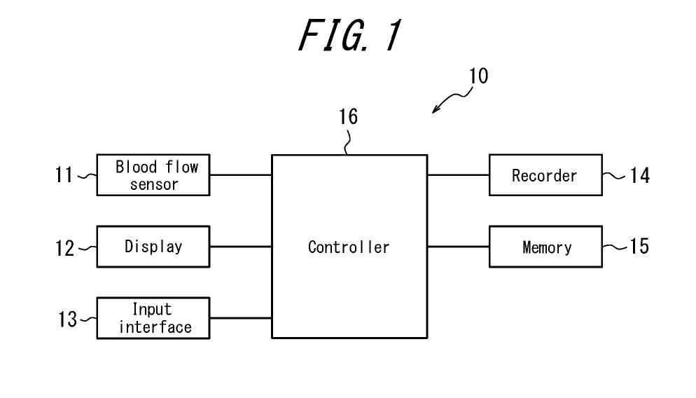

FIG. 1 is a block diagram schematically illustrating the structure of an electronic device according to one of the embodiments of this disclosure. The electronic device 10 may, for example, be configured by a mobile phone. As illustrated in FIG. 1, the electronic device 10 for example includes a blood flow sensor 11, a display 12, an input interface 13, a recorder 14, a memory 15, and a controller 16.

The user uses the electronic device 10 to input an activity record related to the user's activity into the electronic device 10. The user also uses the blood flow sensor 11 provided in the electronic device 10 to measure blood flow information before and after the time pertaining to the input activity record. The electronic device 10 generates a log (data kept as a record) in which the activity record is associated with the blood flow information measured before and after the time of the activity record and records the log in the recorder 14. By the user repeating these operations, the log accumulates in the recorder 14. When the user measures blood flow information using the blood flow sensor 11 before performing a certain activity, then based on the latest measured blood flow information, the electronic device 10 refers to the log and provides the user with activity advice related to the blood flow information.

The blood flow sensor 11 is only one example of a biological sensor. The biological sensor may be any sensor that can measure biological information. This embodiment, as illustrated in FIG. 1, describes the case of the blood flow sensor 11, which is a biological sensor, measuring blood flow information as biological information. Accordingly, the electronic device 10 provides the user with activity advice to improve or maintain the blood flow state.

The blood flow sensor 11 of this embodiment irradiates incident laser light onto a test site such as skin. The blood flow sensor 11 then receives scattered light that is scattered from still body tissue and does not undergo a Doppler shift and also receives scattered light that is scattered by blood and does undergo a Doppler shift. Subsequently, the blood flow sensor 11 calculates the amount of blood flow by detecting the beat due to interference between the scattered light that does not undergo a Doppler shift and the scattered light that does undergo a Doppler shift.

A technique for measuring the amount of blood flow using the Doppler shift is now described. The blood flow sensor 11 that uses the Doppler effect includes an irradiator that irradiates laser light into body tissue of an organism, a light receiver that receives scattered light scattered from body tissue of the organism, and the measurement unit that calculates the amount of blood flow based on the result of measuring the received laser light. The irradiator may, for example, be configured by a laser diode, and the light receiver may be configured by a photodiode.

When the irradiator irradiates laser light onto body tissue of an organism, scattered light that is scattered from moving blood cells undergoes a frequency shift (Doppler shift), due to the Doppler effect, proportional to the speed of travel of the blood cells within the blood. The measurement unit detects the beat signal due to interference between the scattered light from still tissue and the scattered light from moving blood cells. This beat signal represents strength as a function of time. The measurement unit then turns the beat signal into a power spectrum in which power is represented as a function of frequency. In this power spectrum of the beat signal, the Doppler shift frequency is proportional to the speed of blood cells, and the power corresponds to the amount of blood cells. The measurement unit calculates the amount of blood flow by multiplying the power spectrum of the beat signal by the frequency and integrating.

The activity record recorded by the recorder 14 can be a record of any activity, but in this disclosure an activity record of activities related to meals and exercise is described as an example. Accordingly, in this disclosure, the case is described of the user inputting, into the electronic device 10, an activity record related to the content and extent of meals and exercise.

The blood flow sensor 11 for example includes an irradiator and a light receiver. The irradiator irradiates measurement light onto the user's blood flow test site. The irradiator may, for example, be configured by a laser light source that irradiates, as measurement light, laser light with a wavelength that can detect a predetermined component included in blood. An example of such a laser light source is a Laser Diode (LD). The light receiver receives reflected light (detected light) that is reflected from the blood flow test site by the irradiator irradiating the blood flow test site with measurement light. The light receiver is configured by a Photo Diode (PD).

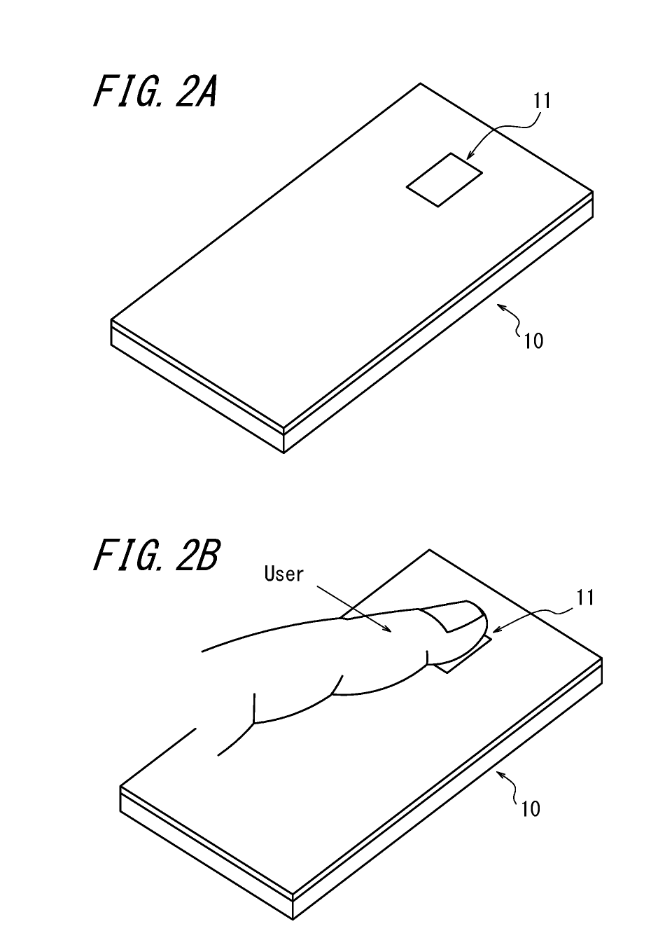

FIGS. 2A and 2B are external views of an example of the electronic device 10 in which the blood flow sensor 11 is mounted. The mobile phone that is the electronic device 10 for example includes the blood flow sensor 11 on the back face, as illustrated in FIG. 2A. The user measures blood flow by pressing the pad of a finger against the blood flow sensor 11, for example as illustrated in FIG. 2B.

In FIG. 1, the display 12 is a display device such as a liquid crystal display, an organic EL display, an inorganic EL display, or the like. The display 12 for example displays the blood flow state based on the blood flow information measured by the blood flow sensor 11. In greater detail, the display 12 for example displays a numerical value related to the amount of blood flow. For example based on the value of the blood flow information measured by the blood flow sensor 11, the display 12 displays whether the blood flow is “thin,” “somewhat thick,” or “thick” using predetermined thresholds as standards. In this way, the display 12 displays the blood flow state with characters, images, numbers, or the like. The display 12 also displays the activity advice calculated by the controller 16, for example as a sentence. A “thick” blood flow refers to a state in which the blood has high viscosity, whereas a “thin” blood flow refers to a state in which the blood has low viscosity.

The input interface 13 accepts operation input from the user and may be configured, for example, using operation buttons (operation keys). The input interface 13 may be configured as a touch panel, a portion of the display 12 may display the input interface 13 that accepts operation input from the user, and this portion may accept touch operation input by the user. The user uses the input interface 13 for example to input the activity record. Details on the activity record and activity plan input by the user are provided below.

The user uses the input interface 13 to input the activity plan. The user for example inputs an activity plan related to a meal, exercise, or the like along with the planned time. The user may input the activity plan related to exercise by inputting the content of a specific exercise as an activity plan.

The user can also use the input interface 13 for example to input information necessary for health management, such as the user's age, height, and weight.

The recorder 14 records a log in which the user's activity record is associated with the biological information measured by the biological sensor before and after the time pertaining to the activity record. In this embodiment, the recorder 14 in particular records a log in which the activity record is associated with the blood flow information measured by the blood flow sensor 11.

The memory 15 may be configured by a semiconductor memory or the like. The memory 15 stores a variety of information, programs for causing the electronic device 10 to operate, and the like. The memory 15 for example stores the activity plan input by the user. The memory 15 also stores information, input by the user, that is necessary for health management. The memory 15 may include the functions of the above-described recorder 14. In other words, in this case, the recorder 14 and the memory 15 are configured integrally.

The controller 16 is a processor that controls overall operations of the electronic device 10. For example when the blood flow sensor 11 has measured the latest blood flow state, the controller 16 provides the user, via the display 12, with activity advice to improve or maintain the blood flow state by referring to the log recorded in the recorder 14 based on the latest blood flow state. At this time, the controller 16 may in particular display activity advice related to the user's activity plan stored in the memory 15. In other words, regarding the user's activity plan stored in the memory 15, the controller 16 may, for example, display the content of a suggested activity.

Next, the activity record that the user inputs using the input interface 13 is described. As an activity record related to a meal, the user for example inputs information related to the content of the meal, the time of the meal, and the like. The content of the meal is the specific content of the meal that was consumed, such as the food that was consumed in the meal, the method of preparing the food, the amount of food, and the like.

The content of the meal can be input in a variety of ways. For example, the user can input the content of the meal by selecting the menu item for the meal from a list of menu items displayed on the display 12 (menu list). The menu list is composed of specific names of menu items, such as “beef curry,” “hamburger,” “salt-grilled Pacific saury,” and the like. The menu list may, for example, be stored in the memory 15 in advance. When the user inputs information by selecting a menu item from the menu list, the controller 16 receives the following as the activity record for the content of the meal: the foods used in the menu item, the method of preparation, and the amount of each food for one person, which are stored in the memory 15. In this way, the user can easily input the activity record.

The user can also input the content of the meal by selecting in accordance with the display on the display 12 for each food. In greater detail, the user for example first operates the input interface 13 to cause the display 12 to display a list of foods (food list). The food list is composed of names of foods, such as “carrot,” “egg,” “Pacific saury,” and the like. The user uses the input interface 13 to select the foods consumed during the meal from the displayed food list. The controller 16 then displays a list related to the method of preparation (method of preparation list) on the display 12. The method of preparation list is composed of the names of methods of preparation, such as “bake,” “boil,” and “steam.” The user uses the input interface 13 to select the method of preparation for the selected food. The controller 16 then displays a list related to the amount of food (amount list) on the display 12. The amount list is composed of units indicating amounts, such as “X pieces,” “X tablespoons,” “X grams,” and the like. The user uses the input interface 13 to input the amount of food by selecting a unit from the amount list and inputting a number for X. By repeating this input operation for each food, the user inputs information related to the meal into the electronic device 10. In this way, the user can input accurate information on a meal as the activity record.

The user can also input the time of the meal into the electronic device 10 by a variety of methods. For example, the user can input the start time and end time of the meal using the input interface 13. The user can also, for example, input the time of the meal by providing input at the start of the meal indicating that the meal has started and providing input at the end of the meal indicating that the meal has ended. The input indicating the start of the meal may, for example, be provided at the start of a meal using a watch function in the electronic device 10 by tapping a button, labeled “meal start,” that is displayed on the input interface 13 of the electronic device 10 configured as a touch panel. The same is also true for the input indicating the end of the meal. The controller 16 associates the information related to the time of the meal input in this way with the content of the meal and receives the result as the activity record related to the meal.

When, in addition to the meal-related activity record input by the user, the blood flow sensor 11 has measured the blood flow information before and after the time pertaining to the activity record, the electronic device 10 may generate a log in which the activity record is associated with the blood flow state that is based on the blood flow information and record the log in the recorder 14. In other words, when the blood flow information is not measured before and after the time pertaining to the activity record, the electronic device 10 need not record the log. In this way, by accumulating the log in which the activity record and the blood flow state are associated, the electronic device 10 can more easily analyze the relationship between changes in the blood flow state and the activities that cause such changes.

With the same method as for an activity record related to a meal, the user can input an activity record related to exercise using the input interface 13. In greater detail, an activity record related to exercise includes the content of the exercise and the extent of the exercise. The content of the exercise is the specific exercise, such as “running” or “futsal.” The extent of the exercise is information such as the length of time the exercise was performed. The length of time the exercise was performed may, for example, be input by the user as a specific numerical value, such as “X minutes,” or be calculated by the controller 16 by the user inputting the start time and end time of the exercise. When the content of the exercise is “running,” for example, the extent of the exercise may also be the distance that was run. When, in addition to the exercise-related activity record that was input, the blood flow sensor 11 has measured the blood flow information before and after the time pertaining to the activity record, the electronic device 10 may record, in the recorder 14, a log in which the activity record is associated with the blood flow state that is based on the blood flow information.

In this embodiment, the activity record related to exercise may further include information related to hydration. Information related to hydration is the amount of water consumed by the user during and after the exercise pertaining to the activity record. In this embodiment, the blood flow information measured as biological information is affected by the state of hydration. Therefore, by including information related to hydration in the activity record, the electronic device 10 can provide advice that is more appropriate for the user.

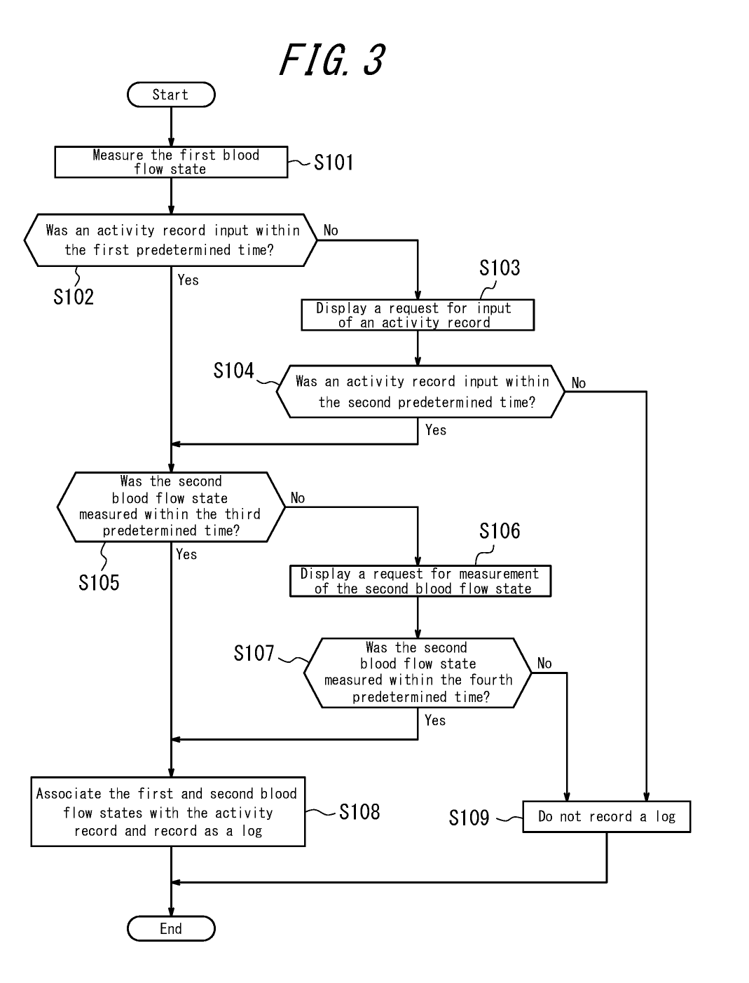

Next, processing to record the log is described. FIG. 3 is a flowchart illustrating an example of processing to record a log in the electronic device 10 of FIG. 1. The flowchart in FIG. 3 illustrates processing to record a log in the case of the user having eaten a meal.

First, the blood flow sensor 11 measures the user's first blood flow state (step S101). The first blood flow state is information indicating the blood flow state before the user eats a meal.

After the blood flow sensor 11 measures the first blood flow state, the controller 16 monitors whether the user has input an activity record within a first predetermined time from measurement of the first blood flow state (step S102). The user for example inputs the activity record using the input interface 13 before or after consuming a meal. When the user has input the activity record related to a meal within the first predetermined time, the controller 16 recognizes the activity record related to a meal (step S102: Yes). Processing then continues to step S105.

On the other hand, when judging that the activity record has not been input within the first predetermined time (step S102: No), the controller 16 displays an input request on the display 12 as a reminder to encourage the user to input the activity record (step S103). The controller 16 then monitors whether the user has input an activity record within a second predetermined time from measurement of the first blood flow state (step S104). The second predetermined time is longer than the first predetermined time.

When the user has input the activity record related to a meal within the second predetermined time, the controller 16 recognizes the activity record related to a meal (step S104: Yes). Processing then continues to step S105.

When the activity record is not input within the second predetermined time (step S104: No), the controller 16 determines not to record the measured first blood flow state as a log (step S109). In this case, the controller 16 terminates the processing flow.

When the activity record is input within the first or second predetermined time after the first blood flow state is measured (step S102: Yes, or step S104: Yes), the controller 16 monitors whether a second blood flow state is measured within a third predetermined time from the meal end time input as the activity record (step S105). The second blood flow state is the blood flow state after the user ate the meal. Accordingly, the second blood flow state changes in accordance with the content of the user's meal.

When the blood flow sensor 11 measures the blood flow state within a third predetermined time after the user finishes the meal, the controller 16 judges that the second blood flow state has been measured (step S105: Yes). Processing then continues to step S108.

On the other hand, when judging that the second blood flow state has not been measured within the third predetermined time (step S105: No), the controller 16 displays a measurement request on the display 12 as a reminder to encourage the user to measure the second blood flow state (step S106). The controller 16 then monitors whether the second blood flow state has been measured within a fourth predetermined time from when the user finished the meal (step S107). The fourth predetermined time is longer than the third predetermined time.

When the blood flow sensor 11 has measured the second blood flow state within the fourth predetermined time, the controller 16 recognizes that the second blood flow state has been measured (step S107: Yes). Processing then continues to step S108.

When the second blood flow state is not measured within the fourth predetermined time (step S107: No), the controller 16 determines not to record the measured first blood flow state as a log (step S109). In this case, the controller 16 terminates the processing flow.

When the second blood flow state was measured within the third or fourth predetermined time after the user finished the meal (step S105: Yes, or step S107: Yes), the controller 16 associates the first blood flow state and the second blood flow state with the activity record and records the result as a log in the recorder 14 (step S108).

As described above, when an activity record and information related to the blood flow states before and after the activity record is gathered, the electronic device 10 associates the activity record with the change in the blood flow state and records the result as a log. In this way, the electronic device 10 can collect a log on the relationship between changes in the blood flow state and the activities that cause the changes. Since the degree of change in the blood flow state differs by user, collecting the log in this way allows the electronic device 10 to provide activity advice based on a particular user's tendencies.

The activity advice provided by the controller 16 is now described. When eating a meal, the user uses the electronic device 10 to measure the blood flow state. As a result, by repeating the process in FIG. 3 described above, the electronic device 10 accumulates the log in the recorder 14. Suppose that in a state in which the log is accumulated in the recorder 14 in this way, the user measures the blood flow state before eating a meal. Further suppose that by user input to the electronic device 10 in advance, eating of a meal has been stored in the memory 15 as the next activity plan for which the blood flow state was measured. At this time, the controller 16 provides activity advice to improve or maintain the blood flow state by referring to the log accumulated in the recorder 14 based on the latest measured blood flow state.

For example, in the log recorded in the recorder 14, suppose that if the blood flow state before the meal was “somewhat thick,” the blood flow state tended to remain “somewhat thick” after the meal when the user consumed meat. Further suppose that if the blood flow state before the meal was “somewhat thick,” the blood flow state tended to be “thin” after the meal when the user consumed fish. In a state in which such a log is recorded, suppose that the blood flow state was “somewhat thick” as a result of the user measuring the latest blood flow state. At this time, the controller 16 for example provides activity advice for improving the blood flow state from “somewhat thick” to “thin.” The controller 16 specifically provides activity advice recommending fish, such as, “Your current blood flow is somewhat thick. A meal based on fish is recommended.”

The change in blood flow state may also differ depending on the method of preparation. Accordingly, in the activity advice, the controller 16 may indicate a recommended method of preparation. For example, in the log recorded in the recorder 14, suppose that if the blood flow state before the meal was “somewhat thick,” the blood flow state tended to change to “thick” after the meal when the user consumed meat that was prepared by being “grilled.” Further suppose that if the blood flow state before the meal was “somewhat thick,” the blood flow state tended to remain “somewhat thick” after the meal when the user consumed meat that was prepared by being “boiled” or “steamed.” In a state in which such a log is recorded, suppose that the blood flow state was “somewhat thick” as a result of the user measuring the latest blood flow state. At this time, the controller 16 for example provides activity advice for maintaining the current blood flow state so that the blood flow state does not degrade from “somewhat thick” to “thick.” The controller 16 specifically provides activity advice recommending a method of preparation that does not cause the blood flow state to degrade easily, such as, “Your current blood flow is somewhat thick. If you are having meat, it is recommended that you have ‘boiled’ or ‘steamed’ meat.”

When the food is meat or fish, the change in blood flow state may also differ depending on the part of the meat. Accordingly, in the activity advice, the controller 16 may indicate a recommended part. The case of consuming pork is described as an example. For example, in the log recorded in the recorder 14, suppose that if the blood flow state before the meal was “somewhat thick,” the blood flow state tended to change to “thick” after the meal when the user consumed “spare ribs.” Further suppose that if the blood flow state before the meal was “somewhat thick,” the blood flow state tended to remain “somewhat thick” after the meal when the user consumed “tenderloin.” In a state in which such a log is recorded, suppose that the blood flow state was “somewhat thick” as a result of the user measuring the latest blood flow state. At this time, the controller 16 for example provides activity advice for maintaining the current blood flow state so that the blood flow state does not degrade from “somewhat thick” to “thick.” The controller 16 specifically provides activity advice recommending a part that does not cause the blood flow state to degrade easily, such as, “Your current blood flow is somewhat thick. If you are having pork, it is recommended that you have tenderloin.”

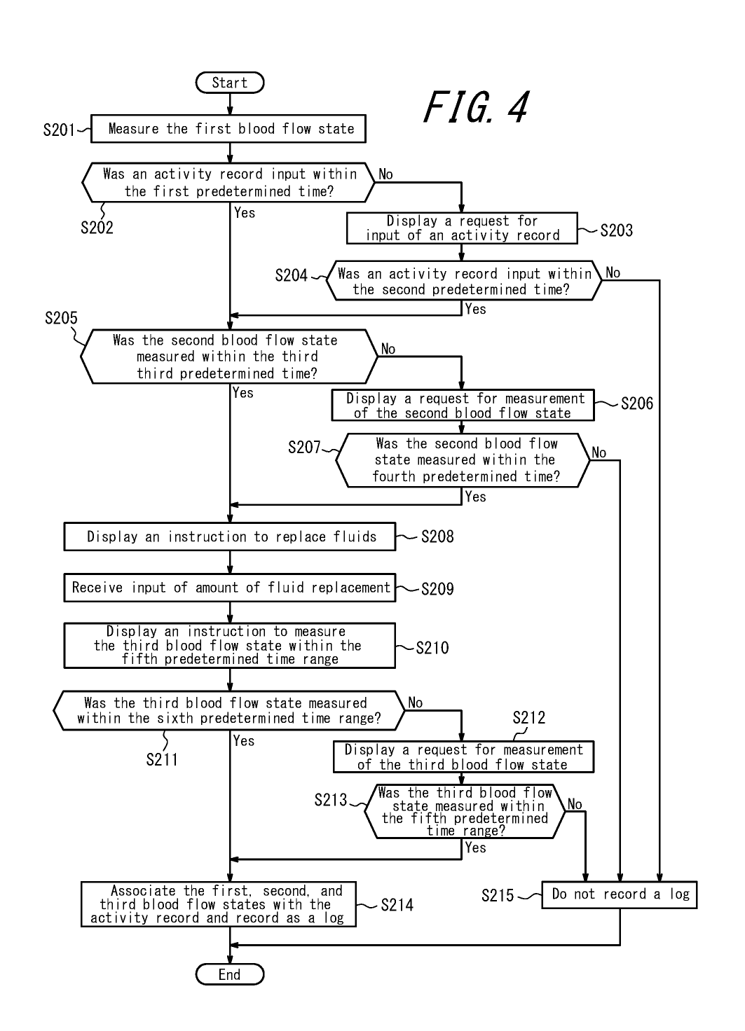

Next, processing to record a log in the case of the user performing exercise is described. FIG. 4 is a flowchart illustrating an example of processing to record a log in the electronic device 10 of FIG. 1.

First, the blood flow sensor 11 measures the user's first blood flow state (step S201). The first blood flow state is information indicating the blood flow state before the user exercises.

After the blood flow sensor 11 measures the first blood flow state, the controller 16 monitors whether the user has input an activity record within a first predetermined time from measurement of the first blood flow state (step S202). When the user has input the activity record related to exercise within the first predetermined time, the controller 16 recognizes the activity record related to exercise (step S202: Yes). Processing then continues to step S205.

On the other hand, when judging that the activity record has not been input within the first predetermined time (step S202: No), the controller 16 displays an input request on the display 12 as a reminder to encourage the user to input the activity record (step S203). The controller 16 then monitors whether the user has input an activity record within a second predetermined time from measurement of the first blood flow state (step S204). The second predetermined time is longer than the first predetermined time.

When the user has input the activity record related to exercise within the second predetermined time, the controller 16 recognizes the activity record related to exercise (step S204: Yes). Processing then continues to step S205.

When the activity record is not input within the second predetermined time (step S204: No), the controller 16 determines not to record the measured first blood flow state as a log (step S215). In this case, the controller 16 terminates the processing flow.

When the activity record is input within the first or second predetermined time after the first blood flow state is measured (step S202: Yes, or step S204: Yes), the controller 16 monitors whether a second blood flow state is measured within a third predetermined time from the exercise end time input as the activity record (step S205). The second blood flow state is the blood flow state after the user exercised. Accordingly, the second blood flow state changes in accordance with the content and extent of the user's exercise. When exercising, the user normally loses water by sweating. Therefore, the second blood flow state tends to be a worse state than the first blood flow state, i.e. a state closer to “thick.”

When the blood flow sensor 11 measures the blood flow state within a third predetermined time after the user finishes exercising, the controller 16 judges that the second blood flow state has been measured (step S205: Yes). Processing then continues to step S208.

On the other hand, when judging that the second blood flow state has not been measured within the third predetermined time (step S205: No), the controller 16 displays a measurement request on the display 12 as a reminder to encourage the user to measure the second blood flow state (step S206). The controller 16 then monitors whether the second blood flow state has been measured within a fourth predetermined time from when the user finished exercising (step S207). The fourth predetermined time is longer than the third predetermined time.

When the blood flow sensor 11 has measured the second blood flow state within the fourth predetermined time, the controller 16 recognizes that the second blood flow state has been measured (step S207: Yes). Processing then continues to step S208.

When the second blood flow state is not measured within the fourth predetermined time (step S207: No), the controller 16 determines not to record the measured first blood flow state as a log (step S215). In this case, the controller 16 terminates the processing flow.

When the second blood flow state was measured within the third or fourth predetermined time after the user finished exercising (step S205: Yes, or step S207: Yes), the controller 16 displays an instruction, on the display 12, for the user to replace fluids (step S208). At this time, the controller 16 may suggest a specific amount of fluid to replace based on information such as the first or second blood flow state.

When the user replaces fluids after confirming the instruction to replace fluids on the display 12, the user uses the input interface 13 to input the amount of fluid replaced as an activity record related to exercise. The controller 16 receives the input amount of fluid replacement (step S209).

Next, the controller 16 displays an instruction, on the display 12, for the user to measure a third blood flow state within a fifth predetermined time range (step S210). The fifth predetermined time range is a time range, after fluid replacement, during which the replaced fluid affects the blood flow. In other words, the fifth predetermined time range is the range of time from when the replaced fluid sufficiently penetrates into the blood until the replaced fluid begins to be lost from the blood. The controller 16 assumes that the user replaced fluid at the time that the amount of fluid replacement was input in step S209 and displays the fifth predetermined time range based on the time at which input of the amount of fluid replacement was received. Accordingly, the third blood flow state indicates the blood flow state as a result of the user replacing fluid after exercising. It is envisioned that due to the effects of exercise, the third blood flow state will be an improvement over the first blood flow state.

Next, the controller 16 monitors whether the third blood flow state has been measured within a sixth predetermined time range (step S211). The sixth predetermined time range is any time range within the fifth predetermined time range.

When the user has the blood flow sensor 11 measure the blood flow state within the sixth predetermined time, the controller 16 judges that the third blood flow state has been measured (step S211: Yes). Processing then continues to step S214.

On the other hand, when judging that the third blood flow state has not been measured within the sixth predetermined time (step S211: No), the controller 16 displays a measurement request on the display 12 as a reminder to encourage the user to measure the third blood flow state (step S212). The controller 16 then monitors whether the third blood flow state has been measured within the fifth predetermined time range (step S213).

When the blood flow sensor 11 has measured the blood flow state within the fifth predetermined time, the controller 16 recognizes that the third blood flow state has been measured (step S213: Yes). Processing then continues to step S214.

When the third blood flow state is not measured within the fifth predetermined time (step S213: No), the controller 16 determines not to record the measured first and second blood flow states as a log (step S215). In this case, the controller 16 terminates the processing flow.

When the third blood flow state was measured within the fifth predetermined time (step S211: Yes, or step S213: Yes), the controller 16 associates the first blood flow state, the second blood flow state, and the third blood flow state with the activity record and records the result as a log in the recorder 14 (step S214).

The log in which the activity record related to exercise is associated with blood flow states does not necessarily need to include the blood flow state after fluid replacement (third blood flow state). For example, when the second blood flow state is measured in the processing illustrated in FIG. 4, if the controller 16 has displayed the instruction to replace fluids (step S208), but the user has not input the amount of fluid replacement (step S209), then the controller 16 may skip the steps related to fluid replacement (step S210 through step S213), associate the first and second blood flow states with the activity record, and record the result as a log in the recorder 14 (step S214).

When inputting the activity record related to exercise, as when inputting the activity record related to a meal, the electronic device 10 associates the activity record with a change in blood flow state and records the result as a log. The degree of change in the blood flow state due to exercise differs by user, but accumulating the log in this way allows the electronic device 10 to provide activity advice based on a particular user's tendencies.

The activity advice provided by the controller 16 with regard to the activity record related to exercise is now described. When exercising, the user uses the electronic device 10 to measure the blood flow state. By repeating the process in FIG. 4 described above, the electronic device 10 accumulates the log in the recorder 14. Suppose that in a state in which the log is accumulated in the recorder 14 in this way, the user measures the blood flow state before exercising. Further suppose that by user input to the electronic device 10 in advance, exercise has been stored in the memory 15 as the next activity plan for which the blood flow state was measured. At this time, based on the latest measured blood flow state, the controller 16 refers to the log accumulated in the recorder 14 and provides activity advice to improve or maintain the blood flow state.

A specific example of the exercise being “running” is described here. For example, in the log recorded in the recorder 14, suppose that if the blood flow state before running was “thick,” the blood flow state tended to change to “somewhat thick” after running for 15 minutes and replacing fluids. In the log recorded in the recorder 14, further suppose that if the blood flow state before running was “thick,” the blood flow state tended to change to “thin” after running for 30 minutes and replacing fluids. In a state in which such a log is recorded, suppose that the blood flow state was “thick” as a result of the user measuring the latest blood flow state. At this time, the controller 16 for example suggests a running time as a guideline for improving the blood flow state. The controller 16 specifically provides activity advice such as, “Your current blood flow is thick. To make your blood flow thin, it is recommended that you run for at least 30 minutes and then replace fluids.”

The change in blood flow state may also differ depending on the amount of fluid replacement after running. Accordingly, in the activity advice, the controller 16 may indicate a recommended amount of fluid replacement. For example, in the log recorded in the recorder 14, suppose that if the blood flow state after running for 30 minutes was “thick,” the blood flow state tended to change to “somewhat thick” after replacing 500 ml of fluids. In the log recorded in the recorder 14, further suppose that if the blood flow state after running for 30 minutes was “thick,” the blood flow state tended to change to “thin” after replacing 750 ml of fluids. In a state in which such a log is recorded, suppose that the blood flow state was “thick” as a result of the user measuring the latest blood flow state after running for 30 minutes. At this time, in step S209 of the flowchart in FIG. 4, the controller 16 for example suggests an amount of fluid replacement as a guideline for improving the blood flow state. The controller 16 specifically provides activity advice such as, “Your current blood flow is thick. To make your blood flow thin, it is recommended that you replace at least 750 ml of fluids.”

As described above, the electronic device 10 records a log, in the recorder 14, in which the user's activity record is associated with the change in blood flow state before and after the activity record. Based on the latest blood flow state, the electronic device 10 provides activity advice. The log recorded in the recorder 14 indicates the tendency for the user's blood flow state to change. Therefore, by referring to the log, the electronic device 10 can provide activity advice based on the user's tendencies. Therefore, it is expected that users who act on the activity advice have a high probability of improving their blood flow state. In this way, the electronic device 10 can provide health support information that is highly useful for the user.

When the user records an activity plan in the memory 15 of the electronic device 10, the electronic device 10 can also provide activity advice on the activity that the user is about to perform. Therefore, the electronic device 10 can provide activity advice that conforms to the user's activities. Accordingly, the electronic device 10 can provide health support information that is even more useful.

The present disclosure is not limited to the above embodiment, and a variety of modifications and changes are possible. For example, the functions and the like included in the various components and steps may be reordered in any logically consistent way. Furthermore, components or steps may be combined into one or divided.

For example, in the above-described embodiment, a mobile phone has been used as an example of the electronic device 10, but the electronic device 10 may be any electronic device. The electronic device 10 may, for example, be any sort of electronic device such as a portable music player, a laptop computer, a tablet, a game device, or the like.

The method of inputting the activity record is not limited to input via the input interface 13. For example, in order to input the content of a meal, the electronic device 10 may be further equipped with a camera, and the user may input the content of the meal using the camera. In greater detail, instead of inputting the content of the meal using the input interface 13, the user can input the content of a meal by photographing the meal with the camera. When the user photographs a meal with the camera, the controller 16 judges the content of the meal included in the photographic image using image recognition. In this way, the user can use the camera to input the content of the meal. By using the camera, the user can input the content of a meal more easily. The controller 16 may transmit the content of the meal photographed with the camera to an external server connected over a network, and the external server may judge the content of the meal included in the photographic image using image recognition and transmit the result of the judgment to the electronic device 10 over the network.

In order to input the extent of exercise, for example, the electronic device 10 may be further equipped with an acceleration sensor. The acceleration sensor detects information related to acceleration, such as the direction and magnitude of acceleration acting on the electronic device 10. Information on the acceleration detected by the acceleration sensor is transmitted to the controller 16, and based on the received information, the controller 16 estimates the operation state of the user who is the owner of the electronic device 10. For example, using the acceleration sensor, the electronic device 10 can estimate the number of steps that the user ran. From the number of steps estimated in this way, the controller 16 may estimate the distance that the user ran and recognize this distance as the extent of running. In this case, the user does not need to input the extent of running him or herself. The controller 16 may transmit information related to the acceleration detected by the acceleration sensor to an external server connected over a network, and the external server may estimate the operation state of the user who is the owner of the electronic device 10 based on the information related to the acceleration detected by the acceleration sensor and transmit the result of the estimation to the electronic device 10 over the network.

When the blood flow sensor 11 measures the latest blood flow state, the controller 16 may refer to past activity records and display the cause of the latest blood flow state on the display 12. For example, suppose that the user measures the blood flow state before eating dinner. At this time, for example when the blood flow state is “thick,” the controller 16 infers the cause behind the blood flow state being “thick” by referring to activity records related to past meals. In greater detail, the controller 16 for example refers to activity records related to breakfast and lunch on the same day and for example infers that the blood flow state is “thick” because of eating meat-centered meals. The controller 16 then displays the inferred cause along with the result of measuring the blood flow state on the display 12. For example, the controller 16 displays a message such as, “Your blood flow is thick due to consuming meat-centered meals today.” By providing such a display, the user can learn the cause of the blood flow state. The controller 16 may, for example, further refer to the activity record related to meals or the activity record related to exercise on the previous day to infer the cause of the blood flow state.

Apart from the log of the activity records, the controller 16 may also refer to any information that affects the change in blood flow state and display activity advice. For example, even when going for the same run, the user's amount of sweat changes due to factors such as the weather, temperature, and humidity. Therefore, the weather, temperature, and humidity affect the change in the user's blood flow state. In this case, the controller 16 may display the activity advice with reference to the weather, temperature, and humidity.

When the controller 16 displays activity advice with reference to the weather, temperature, and humidity, the electronic device 10 may, for example, be further equipped with a position detector, such as a Global Positioning System (GPS), and a communication interface that connects to the Internet or the like. Using the position detector, the electronic device 10 identifies the position of the user who owns the electronic device 10. Via the communication interface, the controller 16 acquires information on the weather, temperature, humidity, and the like at the identified position. The controller 16 can display activity advice with reference to the information acquired in this way. As a result, the electronic device 10 can provide the user with even more useful information.

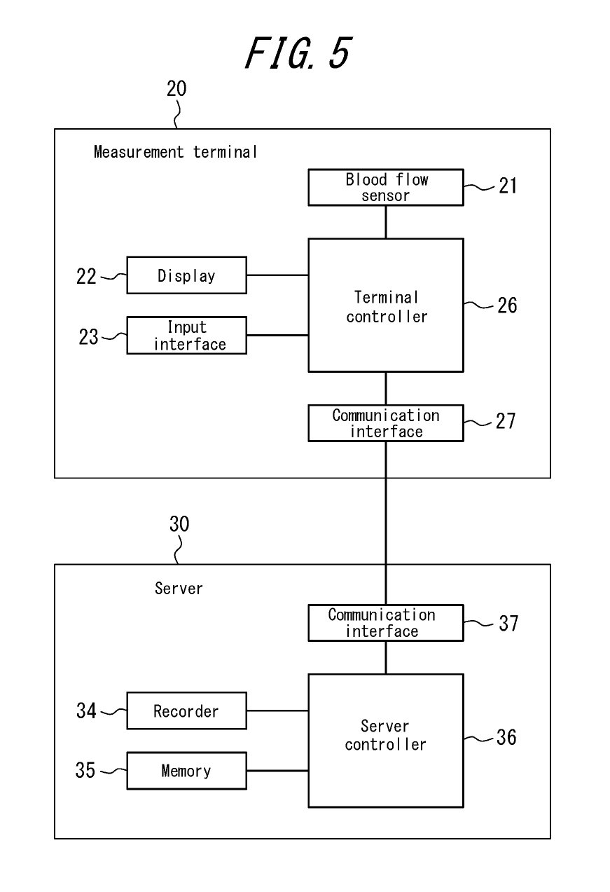

In the above embodiment, all of the following functional units were described as being implemented by one electronic device 10: the blood flow sensor 11, display 12, input interface 13, recorder 14, memory 15, and controller 16. This disclosure is not, however, limited to this aspect. This disclosure may, for example, be implemented as a health support system, as illustrated in FIG. 5, by a measurement terminal 20 that measures blood flow information and displays activity advice and a server 30 that generates activity advice by referring to a log based on the blood flow information.

When this disclosure is implemented in this way as a health support system, the measurement terminal 20 measures the blood flow information in a blood flow sensor 21. The user inputs an activity record, an activity plan, and the like with an input interface 23 of the measurement terminal 20. The measured blood flow information is transmitted from a communication interface 27 to the server 30, which is connected to the measurement terminal 20 over a network constituted by a wired connection, a wireless connection, or a combination thereof. The input activity record and the like are also transmitted to the server 30 via the communication interface 27. On the server 30, the log in which the activity record and the blood flow information are associated are acquired via a communication interface 37 and recorded in a recorder 34. The activity plan acquired via the communication interface 37 is stored in a memory 35. When the latest blood flow information is acquired from the measurement terminal 20 via the communication interface 37, the server controller 36 generates activity advice to improve or maintain the blood flow state and transmits the activity advice to the measurement terminal 20 via the communication interface 37. The server controller 36 thus provides the user with activity advice.

When this disclosure is implemented in this way as a health support system, the measurement terminal 20 can be reduced in size as compared to when all of the functional units are implemented with one electronic device 10.

1. An electronic device comprising:
a biological sensor;
a recorder configured to record a log in which an activity record of a user is associated with biological information measured by the biological sensor before and after a time pertaining to the activity record; and
a controller configured to provide activity advice by referring to the log based on biological information measured by the biological sensor.

2. The electronic device of claim 1, wherein
the biological sensor is a blood flow sensor;
the log is a log in which the activity record is associated with blood flow information measured by the blood flow sensor before and after a time pertaining to the activity record; and
the controller displays activity advice to improve or maintain a blood flow state based on the blood flow information.

3. The electronic device of claim 1, wherein the activity record includes at least one of information related to a meal and information related to exercise.

4. The electronic device of claim 1, further comprising:
a memory configured to store an activity plan of the user; wherein
the controller provides the activity advice in relation to the activity plan.

5. A health support system comprising:
a measurement terminal comprising a biological sensor; and
a server comprising a recorder and a controller, the recorder being configured to record a log in which an activity record related to an activity is associated with biological information measured by the biological sensor before and after a time pertaining to the activity record, and the controller being configured to provide activity advice by referring to the log based on biological information measured by the biological sensor.

6. A health support method comprising:
measuring biological information with a biological sensor;
recording, with a recorder, a log in which an activity record related to a user is associated with the biological information for before and after a time pertaining to the activity record; and
providing, with a controller, activity advice by referring to the log based on measured biological information.
